(19) United States
(12) Patent Application Publication
(10) Pub. No.: US 2023/0015062 A1
(43) Pub. Date: Jan. 19, 2023

(54) GEL COMPOSITIONS FOR MITIGATION OF BURN INJURIES, KITS CONTAINING THE GEL COMPOSITIONS, AND ASSOCIATED METHODS

(71) Applicant: Zaki Yusuf, Louisville, KY (US)

(72) Inventor: Zaki Yusuf, Louisville, KY (US)

(21) Appl. No.: 17/786,198

(22) PCT Filed: Dec. 16, 2020
(86) PCT No.: PCT/US2020/065263

§ 371 Date: Jun. 16, 2022

Related U.S. Application Data
(63) Continuation-in-part of application No. 16/951,335, filed on Nov. 18, 2020.
(60) Provisional application No. 62/950,643, filed on Dec. 19, 2019.

(51) Int. Cl. A61K 9/06; A61K 47/38; A61K 47/12; A61K 31/05; A61K 47/02; A61K 9/00; A61P 17/02

(52) U.S. Cl. CPC A61K 9/06; A61K 9/0014; A61K 31/05; A61K 47/02; A61K 47/12; A61K 47/38; A61P 17/02

(57) ABSTRACT

Compositions and methods are provided for treating burns to minimize hyperkalemia, hyponatremia, blistering, and pain by externally applying gel mixture on burn areas from onset of burn shock. The substrate is a gel mixture containing concentrated sodium ion to create a concentration gradient, allowing in situ diffusion of sodium ion (in vitro) into blister, edema, and extracellular fluids (in vivo) to reduce hyponatremia and (in situ), delivering pH control constituents in vivo to prevent initial acidosis, and minimizing subsequent alkalosis and normalizing SID while simultaneously in situ expelling potassium ions in vitro from the same fluids transdermally while restoring the normal homeostasis condition in the human body. The in situ restoration of homeostasis and electrophysiological conditions also brings blister minimization and pain relief while retarding transcapillary vascular fluid loss to defend kidney and cardiac functions by rectifying transmembrane potential across skeletal, neural, cardiac, and renal cell membranes.

### CROSS REFERENCE TO RELATED APPLICATIONS

This application claims priority to U.S. Provisional Pat. Application No., 62/950,643, filed Dec. 19, 2019, and U.S. Pat. Application No. 16/951,335, filed Nov. 18, 2020, the entire disclosures of which are hereby incorporated by reference.

### TECHNICAL FIELD

The present application is directed to gel compositions for mitigation of burn injuries, to kits containing the gel compositions, and to methods for using the gel compositions.

### BACKGROUND

Burn injuries can range from minor to life threatening and are a serious concern to the health and wellbeing of burn patients. Burns can occur in a variety of everyday situations including, but not limited to, house fires, vehicle accidents, kitchen accidents and electrical malfunctions. Some common sources of burns can include fire, hot objects, cooking utensils, steam, hot liquids, radiation, friction, the sun, electricity or chemicals.

To mitigate pain and promote healing after a burn injury, immediate burn remedies may be critical in the first few moments after the burn injury occurs, when instantaneous care from medical professionals is not possible to prevent skin damage, fluid loss (through blister puncture and/or blister rupture) and organ failure.

Traditionally, remedies such as ice or cold water may be sought for immediate mitigation following burn injuries, but this can lead to detrimental results, e.g., blister formation. Several types of saline solutions mixed with local anesthetics can be somewhat useful for the immediate treatment of burn injuries, but it is difficult to keep these solutions immobilized over the burn area for maximum impact. Additionally, most immediate burn mitigation remedies cannot promote the restoration of homeostasis within the body after the shock of a burn injury.

Burn injury causes severe disruption of normal homeostasis condition (135≤Na⁺≤145 mEq/L, 3.5≤K⁺≤5.0 mEq/L) in the extracellular fluid of human body; and result in malfunction of human organs due to ionic, pH, fluid imbalances in blood plasma/serum and/or extracellular fluid. In general, the delicate and normal homeostatic ion balances and ratios such as Na⁺, K⁺, Ca²⁺ and Cl⁻ and its moderate buffer strength maintains a narrow pH at about 37° C. from 7.35≤pH≤7.45 also governs the electrophysiology of various cells/tissues, ion transport capabilities etc. which in turn controls the delicate kinetic and thermodynamic vulnerability of the entire body including blister formation on skin layer which too are primarily composed of protein and lipid materials.

Moreover, during the burn shock injury, substantial amount of fluid is translocated from the blood vessels due to hypovolemic shock causing the dilution of extracellular fluid resulting in lowering the sodium ion (Na⁺) concentration, causing undesirable hyponatremia (Na⁺≤135 mEq/L) and local initial acidosis (pH≤7.35) in the extracellular fluid. It should be noted that hypovolemic shock also causes blister or edema formation.

Effective pain mitigation and stimulation of healing after a burn injury requires understanding of the optimal homeostasis conditions of various types of cells/tissues (nerve, cardiac, renal etc.), blood, plasma, serum, and extracellular fluid present in the interior of the human body various chemistries of skin inside a human body and rectification of imbalanced homeostasis condition.

Potassium (K⁺) ion is one of the most abundant cations in the intracellular fluid and plays vital role in normal human physiology and electrophysiology. The bulk of total body potassium is intracellular 3500 mEq (-98%), with only approximately 70 mEq (~2%) in the extracellular fluid for a 70 kg human being. This large gradient between intracellular potassium (Kᵢ⁺) (∼120-140 mEq/L) and extracellular potassium (Kₑ⁺) (~4 mEq/L) not only determines the optimal resting membrane potential of most type of cells, they also dictate the depolarization/polarization rates, i.e., the action potentials of the electrically active cell (e.g., cardiac, nerve, skeletal muscles, cardiac muscles and renal etc.) membranes.

The delicate ionic ratios, viz., intracellular potassium (Kᵢ⁺) ion to extracellular potassium (Kₑ⁺) ion ratios (Kᵢ⁺/Kₑ⁺), as well as intracellular sodium (Naᵢ⁺) ion to extracellular sodium (Naₑ⁺), ion ratios (Naᵢ⁺/Naₑ⁺) and their intracellular and extracellular concentrations, i.e., the concentration gradients of the ions and their absolute values are also vitally important for the generation of correct action potentials within the cells; and therefore, are critical for the normal functions of various types of cells. As a result, very small absolute changes in the extracellular potassium ion concentration will have a major effect on this ratio and accordingly, on the function of the all the electrically excitable cells etc. Therefore, potassium and sodium (Na⁺) ions drive the action potentials in various cells by actively crossing the cell membrane and shifting the membrane potentials, which is the difference in electrical potential between the exterior and interior of the cells. As used subsequently herein, the term “Kₑ ⁺” will be denoted simply as “K⁺.”

In addition to being actively transported across cell membranes, potassium ions also move passively (bypassing the gated ion channels) between the extracellular and intracellular compartments. An overload of passive potassium ion transport, such as may be caused by higher levels of extracellular/serum potassium, is capable of raising the resting membrane potentials. Excess potassium ions (≥5.5 mEq/L) in extracellular/serum fluid, known as hyperkalemia, can disrupt the transmembrane potential in cardiac cells that regulate ventricular conduction and contraction. Therefore, the effects of hyperkalemia on cardiac electrophysiology are of greatest concern, because they can cause arrhythmias and death.

The release of potassium ions into the extracellular, blister and intravascular fluid occurs during severe burning due to cell lysis, tissue necrosis and thus releasing exorbitant amount of potassium ions. Additionally, as a result of burn injury, respiratory (e.g., CO₂ inhalation) and metabolic acidosis (lactic acid discharge from muscle cells) may occur in extracellular fluid, i.e., pH drops owing to an increase in the hydrogen ion concentration in blood serum/extracellular fluid. Eventually, this excess hydrogen ion makes its way into the healthy cells in exchange of K⁺ ion release, which in turn, makes their way out in the extracellular fluid and blood serum, thus further complicating hyperkalemia.

Bogart et al. (US 5,271,943) acknowledge that hypertonic sodium chloride formulations are physiologically incompatible on open wound and cytotoxic in nature and can potentially harm or kill healthy cells when they infiltrate in the human body. Therefore, it was recommended by Bogart et al. (US 5,271,943) that hypertonic gel mixture formulations be removed to prevent physiological incompatibility that may kill healthy cells before applying isotonic and hypotonic (≥0.4 wt% and ≤ 0.9 wt%) gel mixture formulations to deliver medications. For large total-burn surface areas (TBSA) of ≥10-20%, the application of hypotonic gel mixture formulations (in US 5,271,943) with lower pH (∼6.8) in open wound may aggravate fluid loss, cause ion and pH imbalances in extracellular fluids undesirable for TBSA of ≥10-20%.

There remain ongoing needs for compositions that mitigate burn injuries and avoid incidental physiological effects caused by ion imbalances that arise after the burn injuries.

### DISCLOSURE OF INVENTION

Accordingly, the present application relates to gel formulations for protecting, treating, and rejuvenating first and second degree burn wounds on exterior layers of skin. The gel formulation can range in pH from 7.01-10.0 at about 37° C. and is intended for external application on closed/unopened burn wounds. The gel formulation provides aqueous, concentrated, sodium chloride (NaCl) and other dissolved aqueous Na⁺ cations of inorganic and organic salts and polyelectrolytes, which diffuse Na⁺ ion in situ across the transdermal concentration gradient. The gel formulation also uses cation exchange resins and anions such as bicarbonate, acetate, citrate, and lactate as pH and SID control ingredients and sodium salt containing buffers. These ingredients are dissolved inside mostly non-crosslinked polymeric substrate gel matrices, where the gel mixture is prepared using sterile and deionized water.

During burn shock, near burn-injured areas, not only does the concentration of K⁺ suddenly jump locally to abnormally high levels (as high as 70 mEq/L or more) but also in the extracellular region, right after cell lysis, tissue necrosis and initial acidosis (due to lactic acid dissociation from the muscle tissues). Further, there is also a surge in fluid volume increase in the extracellular region owing to transcapillary fluid loss from the blood vessels, thus lowering the Na⁺ ion concentration (causing hyponatremia) in the extracellular region. This locally increased concentration of K⁺ can then disperse throughout the vast network of the circulatory system, thus raising the overall concentration of K⁺ ion the extracellular/serum fluids from its normal concentration, causing hyperkalemia. Therefore, it is critical to purge high levels of locally concentrated K⁺ and H⁺ the extracellular fluid transdermally since the excretion of K⁺ through the kidneys takes about four hours and can fatally jeopardize renal functions. Therefore, to contain the K⁺ and H⁺ concentrations in the extracellular fluid to controllable limits, immediate application of this gel mixture formulation over the burn injured areas and their immediate vicinities is critical.

The gel formulation(s) should be applied as early as possible on the exterior of the unopened skin surface in order to facilitate in situ diffusion of the ions transdermally, to regulate and minimize the blister formation, edema, and excess extracellular fluid which take place from the onset of burn shock via capillary loss of plasma/serum. The in situ diffusion of Na⁺ ions from the gel matrix formulation (from in vitro) into the extracellular region (in vivo) helps impede and minimize hyponatremia (Na⁺≤135 mEq/L) while providing counter diffusional expulsion of excess potassium ions from extracellular fluid (in vivo) into the gel matrix (in vitro) for controlling or balancing or reducing and minimizing hyperkalemia in the extracellular and vascular fluid.

In addition, the high pH ranges (pH≥7.55) maintained in the gel matrix formulation, as hydroxyl ions (OH⁻) are released from the dissociation of various sodium salts present in the gel matrix formulation (in vitro), also allows themselves to diffuse via transdermal route in situ, to minimize or prevent initial acidosis condition (as pH drops below 7.35) and to reestablish normal homeostasis (from pH~7.2 to 7.35≤pH≤7.45). Simultaneously, the excess hydrogen (H⁺) ions in the extracellular fluid (in vivo) also move into the gel matrix formulation (in vitro) via in situ counter diffusional ion movement, thus, rectifying the transmembrane potentials of assortment of cells, while simultaneously slowing down/stop vascular transmembrane fluid loss.

With immediate application of a high-pH gel formulation (pH 7.55-10.0) over the burn injured areas, there would be simultaneous local diffusion of Na⁺ and OH⁻ and counter-diffusion of K⁺ and H⁺ in situ, through the transdermal route, swiftly helping to restore homeostatic ion balances, and mitigating initial acidosis and subsequent alkalosis, which, in turn, rectifies the action potentials of various cell functionalities by restoring the normal transmembrane potentials. These actions, including correcting the transmembrane potential of nerve cells, will also reduce pain from the burn injury. This process also provides cardiac and renal protection from the overall increase in the K⁺ concentration by removing excess K⁺ and H⁺ and thus restoring the transmembrane potential of electrically active cells to homeostasis levels.

When gel matrix mixture Formulation (I) as described herein is applied as a pH control agent over burn-injured skin surfaces, the sodium bicarbonate in the gel mixture formulation rectifies local initial acidosis (pH), thereby diminishing hydrogen ion (H⁺) or hydroxyl (OH⁻) ion concentration respectively (in situ), thus preventing potassium ion release from healthy cells and pushing back part of the locally released K⁺ ions in the unharmed cells to prevent subsequent alkalosis (pH≥7.45). Simultaneously, pain is reduced from burn onset or during the hypovolemic phase of burn shock, as the internal electrolyte (K⁺/Na⁺/H⁺) balances are rectified (in situ). Consequently, fluid imbalances are slowed down and/or reversed and subsequently restored to defend, protect, rectify, and restore the organ functionalities by correcting the cell action potentials during the polarization and depolarizations phases of transmembrane ion transport.

This external application of the gel mixture also helps minimize blister formation and subsequent potential blister rupture while also reducing the burden on renal function by creating an alternate path (in situ) for excreting higher levels of K⁺ from the extracellular fluid transdermally. The speedy containment of potassium ion concentration right below 5.5 mEq/L in plasma and extracellular fluid is indispensable to ensure the protection of the vital organs (cardiac and renal functions).

This gel formulation (I) is viscous, with concentrated NaCl and other sodium salts, viz., sodium bicarbonate, sodium carbonate, sodium lactate, sodium citrate and sodium acetate dissolved in deionized and sterile aqueous medium in biodegradable, biopolymers, oligomers and their derivatives as substrate gel matrices dissolved in a pH-controlled condition (pH 7.01-10.0), to (in situ) restore the pH of the extracellular fluid within 7.35≤pH≤7.45. The ionic diffusion of Na⁺ as a form of ion pump helps suppress and minimize blister/edema formation by correcting hyponatremia of the extracellular fluid while suppressing the vascular transcapillary permeability to minimize and control the translocation of serum fluids into the extracellular or interstitial spaces while simultaneously extracting or excreting K⁺ via ionic counter diffusion (in situ) across the transdermal route in the gel matrix (in vitro).

The application of the gel formulation also reduces the requirement of introducing excessive resuscitation fluid via intravenous and enteral routes and thus reduces the negative impacts of introducing excess resuscitation liquid and avoids “fluid creep.” This gel formulation is applicable to thermal and electrical first and second degree burn injuries, but is not appropriate for chemical burns.

Embodiments of this application include methods for preparing gel formulations that can be used as a first response to mitigate first and second degree burn shock by immediate application on the exterior of the injured skin surfaces and their vicinities by minimizing transcapillary fluid loss (and minimizing the proliferation of blisters), hyponatremia (Na⁺≤ 135 mEq/L), hyperkalemia (K+≥5.5 mEq/L), initial acidosis and subsequent alkalosis in order to manage pain, protect cardiac and renal functions.

Embodiments of this disclosure include gel formulations, methods for using the gel formulations, burn kits including the gel formulations, and methods for using the burn kits.

Gel formulations according to embodiments include sodium chloride, sodium bicarbonate, sodium carbonate, sodium lactate, sodium acetate, trisodium citrate a gelling agent, and water from a sterilized and deionized source. The gel formulations may contain only pharmaceutical grade ingredients and may have a total sodium-ion concentration greater than or equal to 154 g/L; a total bicarbonate-ion concentration from 6 × 10⁻⁵ g/L to 17.70 g/L; a yield point of greater than or equal to 1000 poise; and an apparent viscosity from 100 centipoise to150,000 centipoise.

In some embodiments, the gel formulation may have a pH from 7.01 to 10.00 and may include, per liter of the gel formulation at 25° C.: from 80 g to 340 g sodium chloride; from 6 × 10⁻⁵ g to 42 g sodium bicarbonate; from 1.0 × 10⁻⁶ g to 1.3 g sodium carbonate; from 1.6 × 10⁻² g to 156 g sodium lactate; from 1.53 × 10⁻³ g to 82 g sodium acetate; from 0.198 g to 420 g trisodium citrate; and the gelling agent, wherein the gelling agent is selected from the group consisting of hydroxyethyl cellulose, oligomers of cellulose, pectin, carboxymethyl cellulose, guar gum, gum Arabic, and mixtures thereof.

In embodiments, the gel formulation may be a Formulation (I) or a Formulation (II). Generally, the Formulation (I) may be appropriate for use immediately after a burn injury. Generally, the Formulation (II) may be appropriate for use following earlier application of a Formulation (I), particularly to avoid blistering. Other distinguishing characteristics and physiological effects provided by Formulation (I) and Formulation (II) will be described subsequently. Thus, in some embodiments, the gel formulation may be a Formulation (I), having a pH from 7.45 to 10.00 and comprising, per liter of the gel formulation at 25° C.: from 300 g to 340 g sodium chloride; from 3.5 × 10⁻⁴ g to 42 g sodium bicarbonate; from 1 × 10⁻⁶ g to 1.3 g sodium carbonate; from 1.2 × 10⁻¹ g to 156 g sodium lactate; from 1.15 × 10⁻² g to 82 g sodium acetate; and from 1.471 g to 420 g trisodium citrate. In some embodiments, the gel formulation may be a Formulation (II), having a pH from 7.01 to 7.35 and comprising, per liter of the gel formulation at 25° C.: from 80 g to 300 g sodium chloride; from 6.0 × 10⁻⁵ g to 17.7 g sodium bicarbonate; from 1.0 × 10⁻⁶ g to 2.3 × 10⁻⁴ g sodium carbonate; from 1.6 × 10⁻² g to 7.67 × 10⁻² g sodium lactate; from 1.53 × 10⁻³ g to 7.2 × 10⁻² g sodium acetate; and from 0.198 g to 0.954 g trisodium citrate.

In some embodiments, the gel formulation may further include sodium polyacrylate, polyacrylic acid, and less than 300 mg sodium polystyrene sulfonate (Na-PSS; Kayexalate) per liter of the gel formulation. In some embodiments, the gel formulation may further include a pain-relieving agent. Examples of pain-relieving agents include menthol and its derivatives. When menthol is present in the gel formulation, the menthol may have a concentration from 5 g/L to 100 g/L, or from 40 g/L to 50 g/L. In some embodiments, all salts present in the gel formulation are sodium salts, and the gel formulation does not contain any potassium salts or potassium ions. In some embodiments, all salts of the gel formulation are completely dissolved in a gel matrix of the gelling agent and the water.

In one example embodiment, the gel formulation may have a pH from 7.01 to 10.00 and consist exclusively of, per liter of the gel formulation at 25° C.: from 80 g to 340 g sodium chloride; from 6 × 10⁻⁵ g to 42 g sodium bicarbonate; from 1.0 × 10⁻⁶ g to 1.3 g sodium carbonate; from 1.6 × 10⁻² g to 156 g sodium lactate; from 1.53 × 10⁻³ g to 82 g sodium acetate; from 0.198 g to 420 g trisodium citrate; the gelling agent, wherein the gelling agent is selected from the group consisting of hydroxyethyl cellulose, oligomers of cellulose, pectin, carboxymethyl cellulose, guar gum, gum Arabic, and mixtures thereof; and balance water from the sterilized and deionized source.

Features of the gel formulations and their respective ingredients will be described subsequently in detail.

The gel formulations according to embodiments may be used in methods for mitigating a burn injury to a burn victim. Such methods may include applying the gel formulation within 10 minutes of a burn injury on injured skin of the burn victim. The methods may further include spreading the applied gel formulation on the injured skin to prevent loss of vascular fluid into extracellular regions, to expedite sodium-ion transfer across transdermal membranes in vivo, to expel potassium ions across the transdermal membranes in vitro, and to prevent blister formation or proliferation. The methods may further include reapplying fresh gel formulation on the injured skin to maintain high sodium ion concentration gradient across transdermal membranes in vitro to in vivo and high potassium ion concentration gradient across the transdermal membranes in vivo to in vitro.

In the methods for mitigating a burn injury to a burn victim, the gel formulation may have a pH from 7.01 to 10.00 and may include, per liter of the gel formulation at 25° C.: from 80 g to 340 g sodium chloride; from 6 × 10⁻⁵ g to 42 g sodium bicarbonate; from 1.0 × 10⁻⁶ g to 1.3 g sodium carbonate; from 1.6 × 10⁻² g to 156 g sodium lactate; from 1.53 × 10⁻³ g to 82 g sodium acetate; from 0.198 g to 420 g trisodium citrate; and a gelling agent selected from the group consisting of hydroxyethyl cellulose, oligomers of cellulose, pectin, carboxymethyl cellulose, guar gum, gum Arabic, and mixtures thereof. In some embodiments, all salts present in the gel formulation are sodium salts and the gel formulation does not contain any potassium salts or potassium ions. In one example embodiment, the gel formulation may have a pH from 7.45 to 10.00 and consist essentially of, per liter of the gel formulation at 25° C.: from 300 g to 340 g sodium chloride; from 3.5 × 10⁻⁴ g to 42 g sodium bicarbonate; from 1 × 10⁻⁶ g to 1.3 g sodium carbonate; from 1.2 × 10⁻¹ g to 156 g sodium lactate; from 1.15 × 10⁻² g to 82 g sodium acetate; from 1.471 g to 420 g trisodium citrate; a gelling agent selected from the group consisting of hydroxyethyl cellulose, oligomers of cellulose, pectin, carboxymethyl cellulose, guar gum, and gum arabic; and balance water from a sterilized and deionized source.

In the methods for mitigating a burn injury to a burn victim, the gel formulation may include sodium chloride in an amount sufficient to mitigate hyponatremia in blister fluids, extracellular fluids, and blood plasma with simultaneous pain management while restoring sodium/potassium ion imbalances from the burn injury. In the methods for mitigating a burn injury to a burn victim, the gel formulation may include sodium bicarbonate in an amount sufficient to result in mitigating respiratory and metabolic acidosis and SID in blister fluids when pH drops below 7.35 in extracellular fluid and blood plasma with simultaneous pain management while restoring sodium/potassium ion imbalances from the burn injury. In the methods for mitigating a burn injury to a burn victim, the gel formulation may include sodium lactate in an amount sufficient to result in mitigating metabolic acidosis and SID management in blister fluid, extracellular fluid, and blood plasma. In the methods for mitigating a burn injury to a burn victim, the gel formulation may include gelling agent in an amount sufficient to prevent hyperkalemia and acidosis in blister fluids, extracellular fluids, and blood plasma by receiving in vitro excess K⁺ and H⁺ ions from blister fluids, blood plasma, extracellular fluid, while delivering hydroxyl (OH⁻) ions from the gel formulation in vivo into the blister fluid, extracellular fluid, and blood plasma to prevent acidosis and sodium ions from the gel formulation in vivo into the blister fluid, extracellular fluid, and blood plasma to prevent hyponatremia. Thereby, the combination of water, sodium chloride, sodium bicarbonate, sodium lactate, and gelling agent in the gel formulation simultaneously rectifies pH imbalances due to respiratory and metabolic acidosis, expels excess K⁺ ions in vitro, repletes Na⁺ ion deficiency in vivo, restores dynamic physiological Na⁺/K⁺ ion imbalances, and mitigates SID imbalances within blister fluid, extracellular fluid and blood plasma/serum with simultaneous pain management while restoring sodium/potassium ion imbalances from the burn injury.

Further embodiments of this disclosure include burn treatment kits that include a Formulation (I) as previously described and a Formulation (II) as previously described, packaged for use by a person having a burn injury. In this regard the Formulation (I) and the Formulation (II) may be contained in any suitable container such as a bottle or a squeezable tube, for example. The formulations may be further packaged together or separately, optionally with instructions describing their use to mitigate burn injuries. Thus, burn treatment kits according to embodiments may include a first gel formulation that mitigates acidosis, hyponatremia, hyperkalemia when applied following a burn injury; and a second gel formulation that mitigates alkalosis, hyponatremia, hyperkalemia after blister formation is apparent after the burn injury.

In burn treatment kits according to embodiments, the first gel formulation may include, per liter of the first gel formulation at 25° C.: from 300 g to 340 g sodium chloride; from 3.5 × 10⁻⁴ g to 42 g sodium bicarbonate; from 1 × 10⁻⁶ g to 1.3 g sodium carbonate; from 1.2 × 10⁻¹ g to 156 g sodium lactate; from 1.15 × 10⁻² g to 82 g sodium acetate; from 1.471 g to 420 g trisodium citrate; a gelling agent selected from the group consisting of hydroxy ethyl cellulose, oligomers of cellulose, pectin, carboxy-methyl cellulose, guar gum, and gum arabic, and combinations thereof; and water from a sterilized and deionized source. In such embodiments, the first gel formulation has a pH from 7.45 to 10; a total sodium-ion concentration greater than or equal to 154 g/L; a total bicarbonate-ion concentration from 0.01 g/L to 17.70 g/L; a yield point of greater than or equal to 1000 poise; and an apparent viscosity from 100 centipoise to 150,000 centipoise.

In burn treatment kits according to embodiments, the second gel formulation may include, per liter of the second gel formulation at 25° C.: from 80 g to 300 g sodium chloride; from 6.0 × 10⁻⁵ g to 17.7 g sodium bicarbonate; from 1.0 × 10⁻⁶ g to 2.3 × 10⁻⁴ g sodium carbonate; from 1.6 × 10⁻² g to 7.67 × 10⁻² g sodium lactate; from 1.53 × 10⁻³ g to 7.2 × 10⁻² g sodium acetate; from 0.198 g to 0.954 g trisodium citrate; a gelling agent selected from the group consisting of hydroxy ethyl cellulose, oligomers of cellulose, pectin, carboxy-methyl cellulose, guar gum, gum arabic, and combinations thereof; and water from a sterilized and deionized source. In such embodiments, the second gel formulation has a pH from 7.01 to 7.35; a total sodium-ion concentration greater than or equal to 120 g/L; a total lactate-ion concentration from 0.01 g/L to 0.08 g/L; a yield point of greater than or equal to 1000 poise; and an apparent viscosity of less than or equal to 150,000 centipoise.

In some embodiments of the burn treatment kit, all salts present in the first gel formulation and all salts present in the second gel formulation are sodium salts, and the gel formulation does not contain any potassium salts or potassium ions.

Further embodiments are directed to methods for mitigating burn injuries to a human using the burn treatment kit as previously described. Such methods may include applying the first gel formulation to a human having acidosis, hyponatremia, hyperkalemia in extracellular blister fluid within 10 minutes after a burn injury occurs, then applying the second gel formulation to the human having alkalosis, hyponatremia, hyperkalemia to a blister that becomes prominent after ten minutes. During the methods, application of the first gel formulation results in mitigation of acidosis, hyponatremia, hyperkalemia; and application of the second gel formulation results in mitigation of alkalosis, hyponatremia, hyperkalemia after blister formation is visible.

According to embodiments of methods for using the burn kits, the first gel formulation includes, per liter of the first gel formulation at 25° C.: from 300 g to 340 g sodium chloride; from 3.5 × 10⁻⁴ g to 42 g sodium bicarbonate; from 1 × 10⁻⁶ g to 1.3 g sodium carbonate; from 1.2 × 10⁻¹ g to 156 g sodium lactate; from 1.15 × 10⁻² g to 82 g sodium acetate; and from 1.471 g to 420 g trisodium citrate; a gelling agent selected from the group consisting of hydroxy ethyl cellulose, oligomers of cellulose, pectin, carboxy-methyl cellulose, guar gum, and gum arabic, and combinations thereof; and water from a sterilized and deionized source. In such embodiments, the first gel formulation has a pH from 7.45 to 10; a total sodium-ion concentration greater than or equal to 154 g/L; a total bicarbonate-ion concentration from 0.01 g/L to 17.70 g/L; a yield point of greater than or equal to 1000 poise; and an apparent viscosity from 100 centipoise to 150,000 centipoise.

According to embodiments of methods for using the burn kits, the first gel formulation includes, per liter of the second gel formulation at 25° C.: from 80 g to 300 g sodium chloride; from 6.0 × 10⁻⁵ g to 17.7 g sodium bicarbonate; from 1.0 × 10⁻⁶ g to 2.3 × 10⁻⁴ g sodium carbonate; from 1.6 × 10⁻² g to 7.67 × 10⁻² g sodium lactate; from 1.53 × 10⁻³ g to 7.2 × 10⁻² g sodium acetate; from 0.198 g to 0.954 g trisodium citrate; a gelling agent selected from the group consisting of hydroxy ethyl cellulose, oligomers of cellulose, pectin, carboxy-methyl cellulose, guar gum, gum arabic, and combinations thereof; and water from a sterilized and deionized source. In such embodiments, the second gel formulation has a pH from 7.01 to 7.35; a total sodium-ion concentration greater than or equal to 120 g/L; a total lactate-ion concentration from 0.01 g/L to 0.08 g/L; a yield point of greater than or equal to 1000 poise; and an apparent viscosity of less than or equal to 150,000 centipoise. In some embodiments of the methods for using the burn kits, all salts present in the first gel formulation and all salts present in the second gel formulation are sodium salts, and the gel formulations do not contain any potassium salts or potassium ions.

According to embodiments of methods for using the burn kits the first gel formulation comprises sodium chloride in an amount sufficient to mitigate hyponatremia in blister fluids, extracellular fluids, and blood plasma with simultaneous pain management while restoring sodium/potassium ion imbalances from the burn injury. According to embodiments of methods for using the burn kits, the first gel formulation comprises sodium bicarbonate in an amount sufficient to result in mitigating respiratory and metabolic acidosis and SID in blister fluids when pH drops below 7.35 in extracellular fluid and blood plasma with simultaneous pain management while restoring sodium/potassium ion imbalances from the burn injury. According to embodiments of methods for using the burn kits, the first gel formulation comprises sodium lactate in an amount sufficient to result in mitigating metabolic acidosis and SID management in blister fluid, extracellular fluid, and blood plasma. According to embodiments of methods for using the burn kits, the first gel formulation comprises gelling agent in an amount sufficient to prevent hyperkalemia and acidosis in blister fluids, extracellular fluids, and blood plasma by receiving in vitro excess K⁺ and H⁺ ions from blister fluids, blood plasma, extracellular fluid, while delivering hydroxyl ions from the gel formulation in vivo into the blister fluid, extracellular fluid, and blood plasma to prevent acidosis and sodium ions from the gel formulation in vivo into the blister fluid, extracellular fluid, and blood plasma to prevent hyponatremia.

According to embodiments of methods for using the burn kits, the second gel formulation comprises sodium chloride in an amount sufficient to result in mitigating hyponatremia in blister fluid, extracellular fluid and blood plasma with simultaneous pain management while restoring sodium/potassium ion imbalances from the burn injury. According to embodiments of methods for using the burn kits, the second gel formulation comprises sodium lactate and lactic acid in an amount sufficient to result in mitigating alkalosis when plasma pH increase above 7.45 and SID management in blister fluid, extracellular fluid and blood plasma with simultaneous pain management while restoring sodium/potassium ion imbalances from the burn injury. According to embodiments of methods for using the burn kits, the second gel formulation comprises gelling agent in an amount sufficient to result in preventing hyperkalemia (alkalosis) in blister fluid, extracellular fluid and blood plasma by receiving (in vitro) excess K⁺ ion; by delivering the stored sodium ions in vivo in blister fluid, extracellular fluid and blood plasma.

According to embodiments of methods for using the burn kits, in the first gel formulation, the combination of water, sodium chloride, sodium bicarbonate, sodium lactate, and gelling agent in the gel formulation simultaneously rectifies pH imbalances due to respiratory and metabolic acidosis, expels excess K⁺ ions in vitro, repletes Na⁺ ion deficiency in vivo, by restores dynamic physiological Na⁺/K⁺ ion imbalances, and mitigates SID imbalances within blister fluid, extracellular fluid and blood plasma/serum with simultaneous pain management while restoring sodium/potassium ion imbalances from the burn injury. According to embodiments of methods for using the burn kits, in the second gel formulation, the combination of water, sodium chloride, sodium bicarbonate, sodium lactate, and gelling agent simultaneously rectifies pH imbalances due to respiratory and metabolic alkalosis, expels excess K⁺ ions in vitro, repletes Na⁺ ion deficiency in vivo by restoring dynamic physiological Na⁺/K⁺ ion imbalances and SID imbalances within blister fluids, extracellular fluids and blood plasma/serum with simultaneous pain management while restoring sodium/potassium ion imbalances from the burn injury.

Having described now the various gel formulations, methods for using the gel formulations, burn kits including a Formulation (I) and a Formulation (II), and methods for using the burn kits to mitigate burn injuries, the various ingredients, their synergies and interactions, and their physiological effects will now be described in detail, along with protocols for preparing the gel formulations according to embodiments.

With immediate application of Formulation (I) over the burn injured areas, there would be simultaneous local (in situ) diffusion of Na⁺ and OH⁻ ions (in vivo) and counter-diffusion (in vitro) of K⁺ and/or H⁺ ions, and other anion(s) of sodium salt(s) respectively through the transdermal route. These processes swiftly help restore homeostasis (ion balances) from initial acidosis and subsequent alkalosis conditions and, in turn, rectifies the action potentials of various cell functionalities by controlling and correcting the transmembrane potentials and the respective concentrations of extracellular and vascular fluid’s potassium and sodium ion concentrations while reestablishing the regular concentration gradient of K⁺ and Na⁺ ions across the membranes of skeletal, neural, cardiac and other cells. Thus, these overall actions, especially correcting the transmembrane potential of nerve cells, also help reduce pain from the burn injury. The earlier restoration is contingent upon how rapidly the gel mixture formulation is applied across the injured surface areas before the highly concentrated (K⁺≥70 mEq/L) local potassium ions from cell lysis and hydrogen ions from initial acidosis disperses into the vast and intricate network of the circulatory system causing hyperkalemia.

During burn shock, the local concentration of potassium ions suddenly jumps to abnormally high levels (≥70 mEq/L) Soon after cell lysis, tissue necrosis and initial acidosis occur in the extracellular fluid, followed by possible dispersion of potassium ions throughout the circulatory system. Thereby, the overall concentration of K⁺ ion increases in both vascular and extracellular fluids from its normal concentration (K+≥5.5 mEq/L), leading to upsetting the overall homeostasis conditions in the extracellular/plasma fluids. For large total burn surface area (TBSA) injuries, the transcapillary fluid loss from blood vessels also results in large volume of fluid accumulation and weakening of blister membrane, thus, also increasing the viscosity of the serum inside blood vessels, exerting further strain on the cardiac functions.

Therefore, it is critical to immediately and simultaneously retard fluid accumulation in burn injured areas as blister fluid and to purge (in vitro) high levels locally concentrated potassium and hydrogen ions from extracellular fluid (in vivo) and/or to introduce hydroxyl ions in plasma or extracellular fluids (in vivo) via an alternate, shorter, and local routes (in situ) As a basis for comparison, excretion of potassium ion via the kidneys takes a long period (~4 hours), owing to the intricate and extensive network of the circulatory system. This may also jeopardize the renal functions, even to the extent of causing renal failure, owing to the high burden of extracellular fluid imposed by elevated concentrations (K+≥5.5 mEq/L) of potassium ion. Therefore, it is most critical to balance the sodium ion, potassium ion and hydrogen ion concentrations in plasma/extracellular fluids within the homeostatic boundaries by the immediate application of the gel mixture formulation on the burn areas and their vicinities, before the highly concentrated localized potassium and hydrogen ions begin to disperse into the vast network of the circulatory system.

There is another definition that relates to ion balance or imbalance is given by strong ion difference (SID) which is given by the following:

$$\begin{matrix}\text{SID =} \left(\left[{\text{Na}}^{+}\right] + \left[{\text{K}}^{+}\right] + \left[{\text{Mg}}^{2 +}\right] + \left[{\text{Ca}}^{2 +}\right]\right) - \left(\left[{\text{Cl}}^{-}\right] + \left[\left({\text{CH}}_{3} {\text{CHCOO}}^{-}\right)\right]\right) = \\ \left[\text{Dissociated Strong Cations}\right] - \left[\text{Dissociated Strong Anions}\right]\end{matrix}$$

It should be noted that a relatively lower concentration of dissociated bicarbonate ions (HCO₃⁻ ) is present in blood serum/extracellular fluids; therefore, strong ions, i.e., only dissociated ions shows up as significant in the above SID equation (EQN 1). Here it should be noted that SID must be counterbalanced by equal and opposing charges, termed as the effective strong ion difference (SIDe). This measurable difference is referred to as the ‘apparent’ SID (SIDa), with the understanding that not all ions may be accounted for. In healthy humans this number is close to +40 mEq/L. The law of electro-neutrality states that there must be an equal and opposing charge to balance the positive charge, and so the +40 mEq/L is balanced by an equal negative force comprised mostly of weak acids (ATOT).

Therefore, in case of excess chloride ions accumulating in plasma would result in a narrowed strong ion difference (SID) and therefore, resulting in a reduced plasma positive net strong ion charge. When relative plasma positive charge is reduced, as commonly occurs with significant chloride ion loading (reduced SID) As a result, an immediate and compensatory response is the proton or hydrogen-ion generation to assist in restoring the charge equilibrium. Clinicians identify this physiologically disordered process as decreased pH or hyperchloremic acidosis. With hyperchloremic acidosis, a spuriously more negative base deficit or increased base excess, for example by excess lactate (CH₃CHCOO⁻) anion from lactic acid dissociation) as the chloride ion decreases the pH unaccompanied by hypoperfusion and lactic acidemia. Such hyperchloremic condition initiates acidosis in extracellular fluid with lactic acid dissociation, which is also highly undesirable because of excess hydrogen-ion production and the releasing of excess potassium ions from intracellular compartments during homeostasis while the body manages SID in extracellular/plasma fluid.

To circumvent this anomaly, the addition of sodium bicarbonate (NaHCO₃) and other biocompatible sodium salts of organics acid(s) ions (e.g., lactate, acetate, citrate etc.) in the gel mixture formulations also helps transport bicarbonate ions and/or biocompatible organic acid anion(s) and/or their counterpart sodium ions, as well as generated hydroxyl ions. The anions are transported via a diffusion process in situ, across the same transdermal route as a pH control component, to mitigate initial respiratory/metabolic acidosis in the blister/extracellular/plasma/serum fluids.

Therefore, the presence of biocompatible sodium salts of weak organic acids existing as other active ingredients (in vitro) present in the Formulation (I) described in the current disclosure swiftly retards the continuous decrease in pH levels (pH≤7.35) in the extracellular/blister fluids once the anions of weak organic acids and hydroxyl ions diffuse through the transdermal route (in situ) into the extracellular/blister fluid region (in vivo) and/or excess hydrogen ions present in blister/extracellular fluid begins to be purged (in vitro) via counter-diffusion into the gel mixture Formulation (I). Thereby, hyperkalemia, hyponatremia, initial acidosis, blister fluid accumulation, and blister its rupture are swiftly minimized in parallel. Minimization of initial acidosis in turn, reduces potassium-ion release from healthy cells into extracellular/blister fluids from the beginning of burn injury.

For thermal and electrical burn injuries, it is indispensable also to initiate immediate pH stabilization and expulsion of the excess, highly localized and concentrated potassium ions from blister/extracellular fluids transdermally (in situ). Transdermal expulsion bypasses the renal route and thus minimizes blister formation and undesirable blister rupture, safeguarding both renal and cardiac functions. At the same time, (in vitro) the potassium ions are purged to within the applied formulation gel mixture over the skin surface by simultaneously suppressing the initial acidosis (pH≤7.35), checking the subsequent alkalosis (pH≥7.45) and bringing back the pH of extracellular and plasma fluid within the homeostasis ranges (7.35≤pH≤7.45).

Conversely, burn victims with pre-existing alkalosis or delayed applications may require lower pH control (~pH 7.01≤pH≤7.35) ingredients and would require Formulation (II) described subsequently in this application. However, Formulation (II) may be used also when initial acidosis is replaced with subsequent alkalosis sometime after the burn injury has occurred and when local extracellular/blister fluids have already accumulated severe levels of potassium ions after the delay in applying the Formulation (I) at the onset of burn injury. Approximate pH values of different ingredients in the formulations according to this disclosure are provided in Tables 1 and 2.

Table 1

Approximate pH Values versus Various Sodium Salt of Weak Acid Concentration. Note: Atmospheric CO₂ dissolves into gel mixture formulation and may significantly reduce the overall pH of the mixture over time ((Without the use of Activity Coefficients)

| Weak Acid Sodium Salt | Eq/L | pH | mg/L |
|---|---|---|---|
| Sodium Bicarbonate (NaHCO₃) Mol Wt.: 84.0 pKa: 6.35 | 5.00E-01 | 10.02 | 42000.00 |
| | 4.50E-03 | 9.00 | 378.00 |
| | 1.40E-03 | 8.75 | 117.60 |
| | 2.00E-04 | 8.32 | 16.80 |
| | 4.50E-05 | 7.99 | 3.78 |
| | 3.00E-05 | 7.90 | 2.52 |
| | 2.00E-05 | 7.81 | 1.68 |
| | 1.00E-05 | 7.65 | 0.84 |
| Sodium Acetate (CH₃COONa) Mol Wt.: 82.0 pKa: 4.76 | 1.00E+00 | 9.38 | 82000.00 |
| | 1.00E-01 | 8.88 | 8202.36 |
| | 1.00E-02 | 8.38 | 820.24 |
| | 2.00E-03 | 8.03 | 164.05 |
| | 3.00E-04 | 7.62 | 24.61 |
| | 2.00E-04 | 7.53 | 16.40 |
| | 4.00E-05 | 7.18 | 3.28 |
| Sodium Lactate (C₃H₅O₃Na) Mol Wt.: 112.0 pKa: 3.86 | 1.40E+00 | 9.00 | 156800.00 |
| | 1.00E-01 | 8.43 | 11200.00 |
| | 1.50E-02 | 8.02 | 1680.00 |
| | 6.00E-03 | 7.82 | 672.00 |
| | 3.50E-03 | 7.70 | 392.00 |
| | 1.50E-03 | 7.52 | 168.00 |
| | 2.00E-03 | 7.58 | 224.00 |
| | 2.50E-03 | 7.36 | 280.00 |
| Trisodium Citrate (C₆H₅O₇Na₃) Mol Wt.: 258.1 pKa: 3.14 | 1.64E+00 | 8.68 | 423218.40 |
| | 1.00E+00 | 8.57 | 258060.00 |
| | 1.65E-01 | 8.18 | 42579.90 |
| | 1.00E-01 | 8.07 | 25810.00 |
| | 5.00E-02 | 7.92 | 12905.00 |
| | 1.00E-03 | 7.07 | 258.06 |
| | 5.00E-04 | 6.92 | 129.03 |
| | 3.00E-04 | 6.81 | 77.42 |
| | 2.00E-04 | 6.72 | 51.62 |

Table 2

Examples of Concentrated Sodium Bicarbonate and Sodium Carbonate Buffers at Different Ratios to Create a Wider pH Range (~9-10) in Ideal Solution Scenario (Without the use of Activity Coefficients)

| Eq/L | Eq/L | mg/L | mg/L | |
|---|---|---|---|---|
| NaHCO₃ | Na₂CO₃ | NaHCO₃ | Na₂CO₃ | pH |
| 3.33E-01 | 1.67E-01 | 27973.68 | 17691.97 | 10 |
| 3.58E-01 | 1.42E-01 | 30034.56 | 15088.56 | 9.9 |
| 3.80E-01 | 1.20E-01 | 31900.77 | 12729.98 | 9.8 |
| 3.99E-01 | 1.00E-01 | 33556.10 | 10636.48 | 9.7 |
| 4.17E-01 | 8.31E-02 | 34997.42 | 8811.76 | 9.6 |
| 4.31E-01 | 6.84E-02 | 36231.97 | 7246.34 | 9.5 |
| 4.44E-01 | 5.59E-02 | 37274.23 | 5921.55 | 9.4 |
| 4.54E-01 | 4.54E-02 | 38142.91 | 4813.27 | 9.3 |
| 4.63E-01 | 3.67E-02 | 38858.47 | 3895.04 | 9.2 |
| 4.70E-01 | 2.96E-02 | 39441.29 | 3140.35 | 9.1 |
| 4.75E-01 | 2.38E-02 | 39910.39 | 2524.14 | 9.0 |
| 4.80E-01 | 1.91E-02 | 40282.78 | 2023.70 | 8.9 |

For severely burned patients with high TBSA (>20%), to maintain constant sodium ion pump, the initial concentration for sodium ion in the gel mixture formulation according to this disclosure could be as high as M = 5.15 Eq/L (for Na⁺ ion) at t = 0. Such initial concentration of sodium ions imposes ion pumping for faster diffusion of sodium ions into the extracellular fluid as the in vivo (blister/extracellular/plasma) concentration of sodium ion rapidly falls much below ~135 mEq/L due to the transcapillary fluid loss. In turn, hyponatremia is averted. On the other hand, the initial and local concentration of potassium ion in the extracellular/blister fluid (in vivo) is from M_{K}⁺≥5.5 mEq/L to as high as M_{K}⁺≥70 mEq/L. This highly localized concentration in the blister fluid near burn injured areas is excreted or expelled out from the blister and extracellular fluid (in vivo) transdermally, before the potassium ions can disperse themselves away from the burn shock region and its vicinities along the long network of the circulatory system. Initially, the outside (in vitro) concentration of potassium ion, M⁺_{K}(in vitro) = 0 mEq/L at t = 0 in the gel mixture formulation. The transport of bicarbonate ions from sodium bicarbonate, and of other anions from organic sodium salts of weak acids and their surrogate ions such as hydrogen ions, hydroxyl ions, and lactate ions, also helps prevent initial acidosis and SID imbalance.

### EXAMPLES

### Purpose of Aqueous Gel Matrix Formulation

Create an aqueous gel matrix reservoir for sodium chloride (NaCl) in dissolved ionic form, i.e., to create concentrated sodium ion pump, potassium ion sanctuary, hydroxyl/hydrogen ion pump, create a reservoir to dissolve other sodium salts of several other weak organic acids for pH and SID stabilization (in vivo) for their in situ transport through the transdermal route via diffusion.

### Example 1

### Preparation of Exemplary Formulation (I)

An exemplary Formulation (I) is prepared by first transferring 1.0 liter of deionized water in a 2-liter Erlenmeyer flask and cap it with cotton wool. Place the Erlenmeyer flask in an autoclave to sterilize the DI water at 125° C. for more than 15 minutes. Allow the autoclave to cool down to lower the pressure to normal atmospheric pressure and then open the autoclave lid while wearing thermally insulated gloves (appropriate safety measures) and other personal protective equipment (e.g., safety glasses). Bring the Erlenmeyer flask containing sterilized water out of the autoclave. Place the Erlenmeyer flask in UV radiation chamber and irradiate the water filled Erlenmeyer flask. Bring out the Erlenmeyer flask and remove its cotton wool cap. Transfer the steam sterilized Erlenmeyer flask over a magnetically stirred hot plate and continue to maintain 50° C. temperature inside the flask.

Then, weigh 3.78 g of pharmaceutical grade sodium bicarbonate (NaHCO₃) on a watch glass. Add this measured amount of sodium bicarbonate into the Erlenmeyer flask and turn on the magnetic stirrer for continued mixing and maintain 50° C. temperature until dissolved. Weigh 350.00 g of pharmaceutical grade sodium chloride (NaCl) on a watch glass. Transfer the measured amount of sodium chloride into the Erlenmeyer flask and continue with the mixing over a magnetically stirred hot plate and maintain 50° C. temperature until dissolved. Weigh 25.00 g of pharmaceutical grade sodium lactate (CH₃CH(OH)COO^{—}Na⁺) on a watch glass. Transfer the measured amount of sodium lactate into the Erlenmeyer flask and continue with the mixing over a magnetically stirred hot plate and maintain 50° C. temperature until dissolved. Remove the magnets from Erlenmeyer flask and place a high-speed mixer with a double impeller blade arrangement (e.g., NovAspestic high-speed mixer) inside the Erlenmeyer flask. Turn on the mixer and maintain at least 1000 rpm speed while ensuring that air does not become entrained into the solution. Transfer the solution containing dissolved sodium bicarbonate, sodium lactate and sodium chloride in a beaker. Continue to maintain higher temperature inside a (1.5-2.0) liter beaker which is also placed over a hot plate.

Then weigh 40 g of pharmaceutical grade hydroxyethyl cellulose on a watch glass. Transfer the weighed hydroxyethyl cellulose slowly into the beaker and continue with the mixing for minimum of 30 minutes over the hot plate while maintaining 50° C. temperature at 4000 rpm speed for 30 minutes or until dissolved. If required, increase the mixer speed to expedite the complete dissolution of hydroxyethyl cellulose in water without entraining air. Take a sample of the mixer to determine the viscosity of the mixture at 25° C. Once the viscosity is in the range 1000-150,000 cP, stop the mixing. If the viscosity is below 1000 cP, add a small amount of hydroxyethyl cellulose and repeat the mixing steps. Place the beaker in a water bath and continue mixing until the temperature reaches 25° C. Use a pH probe in the mixer to determine the pH of the mixture at 25° C. If the pH reaches 7.8-10 range, stop mixing and bottle the mixture, else add additional sodium bicarbonate until the pH of the mixture reaches 7.8-10 range. The order of addition of ingredients may be changed to facilitate dissolution time.

### Example 2

### Preparation of Formulation (II)

An exemplary Formulation (II) is prepared by first transferring 1 liter of deionized water in a 2 liter Erlenmeyer flask and capping the flask with cotton wool. Place the Erlenmeyer flask in an autoclave to sterilize the DI water at 125° C. for more than 15 minutes. Allow the autoclave to cool down to lower the pressure to normal atmospheric pressure and then open the autoclave lid while wearing thermally insulated gloves and other personal protective equipment. Bring the Erlenmeyer flask containing sterilized water out of the autoclave. Place the Erlenmeyer flask in UV radiation chamber and irradiate the water filled Erlenmeyer flask. Bring out the Erlenmeyer flask from UV irradiation chamber and remove its cotton wool cap. Transfer the steam sterilized Erlenmeyer flask over a magnetically stirred hot plate and continue to maintain 50° C. temperature inside the flask.

Weigh pharmaceutical grade sodium bicarbonate (NaHCO₃) on a watch glass. Add the measured amount of sodium bicarbonate into the Erlenmeyer flask and turn on the magnetic stirrer for continued mixing and maintain 50° C. temperature until dissolved. Weigh pharmaceutical grade sodium chloride (NaCI) on a watch glass. Transfer the measured amount of sodium chloride into the Erlenmeyer flask and continue with the mixing over a magnetically stirred hot plate and maintain 50° C. temperature until dissolved. Weigh pharmaceutical grade sodium lactate (CH₃CH(OH)COO^{—}Na⁺) on a watch glass. Transfer the measured amount of sodium lactate into the Erlenmeyer flask and continue with the mixing over a magnetically stirred hot plate and maintain 50° C. temperature until dissolved. Then remove the magnets from Erlenmeyer flask and place a high-speed mixer with a double impeller blade arrangement (e.g., NovAspestic high-speed mixer) inside the Erlenmeyer flask. Turn on the mixer and maintain at least 1000 rpm speed while ensuring that air is not entrained into the solution. Transfer the solution containing dissolved sodium bicarbonate, sodium lactate, and sodium chloride in a (1.5-2.0) liter beaker. Continue to maintain higher temperature inside a (1.5-2.0) liter beaker which is also placed over a hot plate and maintain at least 1000 rpm speed.

Then, weigh 40 g of pharmaceutical grade hydroxyethyl cellulose on a watch glass. Transfer the weighed hydroxyethyl cellulose slowly into the beaker and continue with the mixing for minimum of 30 minutes over the hot plate while maintaining 50° C. temperature and continuously increase the speed to 4000 rpm speed or more for 30 minutes or until dissolved. If required increase the speed of the mixer to expedite the complete dissolution of hydroxyethyl cellulose in water without entraining air. To enhance better dissolution of hydroxyethyl cellulose in water, decrease the temperature of the mixture by stopping the heat. After complete dissolution of hydroxyethyl cellulose, take a sample from the mixer to determine the viscosity of the mixture at 25° C. Once the viscosity reaches between 1000-150,000 cP stop the mixing. If the viscosity is below 1000 cP, add small amount of hydroxyethyl cellulose and repeat the mixing steps. Place the beaker in water bath and continue mixing until the temperature reaches 25° C. Use a pH probe in the mixer to determine the pH of the mixture at 25° C. Now slowly add small amount of lactic acid to the mixture while vigorously stirring and wait 30 minutes to allow complete mixing and simultaneously take the pH reading at 25° C. If the pH reaches 7.01-7.2 range, stop mixing and bottle the mixture, else add additional lactic acid until the pH of the mixture reaches 7.01-7.2 range.

### Example 3

Various non-limiting examples of Formulations (I) and (II) that are prepared according to Examples 1 or 2, or according to other embodiments of this disclosure, are provided in tabular form in Tables 3 and 4.

Table 3

Exemplary Compositions of Formulation (I). Except where indicated otherwise, ingredient amounts are reported in grams of ingredient per liter of Formulation (I)

| Ingredient | FORMULATION (I) | | | | |
|---|---|---|---|---|---|
| | I-A | I-B | I-C | I-D | I-E |
| DI Water | 1000 | 1000 | 1000 | 1000 | 1000 |
| Sodium Chloride | 300 | 300 | 300 | 300 | 300 |
| Sodium Bicarbonate | 17.69 | 0.38 | 0.0378 | 0.00168 | 0.00109 |
| Sodium Carbonate | 27.93 | — | — | — | — |
| Sodium Lactate | 156 | 156 | 14.5 | 0.69 | 0.39 |
| Sodium Acetate | 82 | 8.2 | 3.7 | 0.16 | 0.05 |
| Trisodium Citrate | 420 | 420 | 42 | 25 | 9 |
| Gelling Agents* | >100.18 | >94.23 | >68.01 | >66.29 | >65.47 |
| Sodium Polyacrylate (ppm) | ≤ 300 | ≤ 300 | ≤ 300 | ≤ 300 | ≤ 300 |
| Na Polystyrene Sulfonate (ppm) | ≤ 300 | ≤ 300 | ≤ 300 | ≤ 300 | ≤ 300 |
| TOTAL | ∼2103.8 | ∼1978.81 | ∼1428.25 | ∼1392.14 | ∼1374.91 |
| Property | PROPERTIES | | | | |
| | I-A | I-B | I-C | I-D | I-E |
| pH | 10 (Buffered) | 9 | 8.5 | 8 | 7.7 |
| Viscosity (cP @ 20° C.) | 100-150,000 | 100-150,000 | 100-150,000 | 100-150,000 | 100-150,000 |
| Injury Type | Severe & Deep | Severe & Deep | Severe & Deep | Severe | Severe or Mild |
| Time of Application | Immediately | Immediately | Immediately | Immediately | Immediately |
| Type of Patient | Normal | Normal | Normal | Normal | Normal |
| Treatment | PHYSIOLOGICAL CONDITIONS | | | | |
| | I-A | I-B | I-C | I-D | I-E |
| Sodium-ion Deficiency | Hyponatremia | Hyponatremia | Hyponatremia | Hyponatremia | Hyponatremia |
| Potassium-Ion proliferation | Hyperkalemia | Hyperkalemia | Hyperkalemia | Hyperkalemia | Hyperkalemia |
| Physiological pH Condition | Acidosis | Acidosis | Acidosis | Acidosis | Acidosis |
| Total Injured Burn Area | ≥ 20% | ≥ 20% | ≥ 10%-20% | ≤ 10% | ≤ 10% |

* The gelling agents include a total amount of one or more of hydroxyethyl cellulose, cellulose oligomers, carboxymethylcellulose and/or gum arabic

Table 4

Exemplary Compositions of Formulation (II). Except where indicated otherwise, ingredient amounts are reported in grams of ingredient per liter of Formulation (II)

| Ingredient | FORMULATION (II) | | | | |
|---|---|---|---|---|---|
| | II-A | II-B | II-C | II-D | II-E |
| DI Water | 1000 | 1000 | 1000 | 1000 | 1000 |
| Sodium Chloride | 350 | 200 | 260 | 350 | 300 |
| Sodium Bicarbonate | 6.05 × 10⁻⁵ | 6.72 × 10⁻⁵ | 8.15 × 10⁻⁵ | 1.01 × 10⁻⁴ | 1.22 × 10⁻⁴ |
| Sodium Carbonate | — | — | — | — | — |
| Sodium Lactate | 1.68 × 10⁻² | 1.96 × 10⁻² | 2.46 × 10⁻² | 3.14 × 10⁻² | 3.92 × 10⁻² |
| Sodium Acetate | 1.56 × 10⁻³ | 1.80 × 10⁻³ | 1.2.30 × 10⁻³ | 2.87 × 10⁻³ | 3.61 × 10⁻³ |
| Trisodium Citrate | 0.21 | 0.24 | 0.3 | 0.37 | 0.46 |
| Gelling Agents* | ≥68.15 | ≥60.64 | ≥63.13 | ≥68.15 | ≥65.1 |
| Sodium Polyacrylate (ppm) | ≤ 300 | ≤ 300 | ≤ 300 | ≤ 300 | ≤ 300 |
| Na Polystyrene Sulfonate (ppm) | — | — | ≤ 300 | — | — |
| Total | ∼1431 | ∼1273 | ∼1326 | ∼1431 | ∼1370 |
| Property | PROPERTIES | | | | |
| | II-A | II-B | II-C | II-D | II-E |
| pH | 7.02 | 7.05 | 7.1 | 7.15 | 7.2 |
| Viscosity (cP @ 20° C.) | 100-150,000 | 100-150,000 | 100-150,000 | 100-150,000 | 100-150,000 |
| For Injury Type | Severe | Severe & Deep | Severe & Deep | Severe | Severe & Deep |
| Time of Application | Delayed | Delayed | Delayed | Delayed | Delayed |
| Type of Patient | Alkalosis Patient | Alkalosis Patient | Alkalosis Patient | Alkalosis Patient | Alkalosis Patient |
| Condition | PHYSIOLOGICAL CONDITIONS | | | | |
| | II-A | II-B | II-C | II-D | II-E |
| Sodium ion deficiency | Hyponatremia | Hyponatremia | Hyponatremia | Hyponatremia | Hyponatremia |
| Potassium ion proliferation | Hyperkalemia | Hyperkalemia | Hyperkalemia | Hyperkalemia | Hyperkalemia |
| pH Condition | Alkalosis | Alkalosis | Alkalosis | Alkalosis | Alkalosis |
| Total Injured Burn Area | ≥ 10% | ≥ 10% | ≥ 20% | ≥ 20% | ≥ 20% |

* The gelling agents include a total amount of one or more of hydroxyethyl cellulose, cellulose oligomers, carboxymethylcellulose and/or gum arabic

### Example 4

In this example, a gel formulation according to the present disclosure was applied to a small-area burn injury on the back side of an injured patient’s left palm within a minute of the injury. During this incident, while frying chicken over a saucepan, a tablespoon of boiling hot butter splashed on the backside of patient’s palm. Immediately, the patient experienced excruciating pain around burn injured areas and its vicinities. The hot butter eventually flowed down toward the left index finger and also caused burning sensation there.

Within one minute of the injury, the patient applied a gel formulation according to the present disclosure on the burn injured areas and its vicinities. A few minutes later, after the application of the formulation, only a faint reddish color appeared on the upper part of the index finger. After the application of the gel formulation, the pain rapidly started to subside. After about 20-45 minutes, nearly all pain had stopped and no blister had formed. The following morning, the patient became oblivious to the pain from previous day’s injury. After a few days, the patient noticed dead skin appearing on the burned areas on the back side of the palm.

Because the burn injury area was less than 10% of TBSA, there was minimal metabolic acidosis and, accordingly, a Formulation (I) was used on the burn injured areas. The composition of this formulation included 340 g/Liter sodium chloride and 3.8 mg/Liter sodium bicarbonate. The gel mixture had a pH of about 8.0. This example evidences that application of the gel formulation according to this disclosure at the onset of a burn injury can prevent blister formation and greatly reduce pain intensity and duration associated with thermal burn injuries.

1. A gel formulation comprising:
sodium chloride;
sodium bicarbonate;
sodium carbonate;
sodium lactate;
sodium acetate;
trisodium citrate;
a gelling agent; and
water from a sterilized and deionized source,
wherein the gel formulation contains only pharmaceutical grade ingredients and has:
a total sodium-ion concentration greater than or equal to 154 g/L; and
a total bicarbonate-ion concentration from 6 × 10⁻⁵ g/L to 17.70 g/L.

2. The gel formulation of claim 1, having a pH from 7.01 to 10.00, a yield point of greater than or equal to 1000 poise, and an apparent viscosity from greater than 100 centipoise to 150,000 centipoise, the gel formulation comprising, per liter of the gel formulation at 25° C.:
from 80 g to 340 g sodium chloride;
from 6 × 10⁻⁵ g to 42 g sodium bicarbonate;
from 1.0 × 10⁻⁶ g to 1.3 g sodium carbonate;
from 1.6 × 10⁻² g to 156 g sodium lactate;
from 1.53 × 10⁻³ g to 82 g sodium acetate;
from 0.198 g to 420 g trisodium citrate; and
the gelling agent, wherein the gelling agent is selected from the group consisting of hydroxyethyl cellulose, oligomers of cellulose, pectin, carboxymethyl cellulose, guar gum, gum Arabic, and mixtures thereof.

3. The gel formulation of claim 2, having a pH from 7.45 to 10.00 and comprising, per liter of the gel formulation at 25° C.:
from 300 g to 340 g sodium chloride;
from 3.5 × 10⁻⁴ g to 42 g sodium bicarbonate;
from 1 × 10⁻⁶ g to 1.3 g sodium carbonate;
from 1.2 × 10⁻¹ g to 156 g sodium lactate;
from 1.15 × 10⁻² g to 82 g sodium acetate; and
from 1.471 g to 420 g trisodium citrate.

4. The gel formulation of claim 2, having a pH from 7.01 to 7.35 and comprising, per liter of the gel formulation at 25° C.:
from 80 g to 300 g sodium chloride;
from 6.0 × 10⁻⁵ g to 17.7 g sodium bicarbonate;
from 1.0 × 10⁻⁶ g to 2.3 × 10⁻⁴ g sodium carbonate;
from 1.6 × 10⁻² g to 7.67 × 10 ² g sodium lactate;
from 1.53 × 10⁻³ g to 7.2 × 10⁻² g sodium acetate;
from 0.198 g to 0.954 g trisodium citrate.

5. The gel formulations of claim 1, wherein all salts present in the gel formulation are sodium salts and the gel formulation does not contain any potassium salts or potassium ions.

6. The gel formulation of claim 1, wherein all salts of the gel formulation are completely dissolved in a gel matrix of the gelling agent and the water.

7. The gel formulation of claim 1, further comprising a pain-relieving agent selected from menthol and derivatives of menthol.

8. The gel formulation of claim 1, having a pH from 7.01 to 10.00 and consisting of, per liter of the gel formulation at 25° C.:
from 80 g to 340 g sodium chloride;
from 6 × 10⁻⁵ g to 42 g sodium bicarbonate;
from 1.0 × 10⁻⁶ g to 1.3 g sodium carbonate;
from 1.6 × 10⁻² g to 156 g sodium lactate;
from 1.53 × 10⁻³ g to 82 g sodium acetate;
from 0.198 g to 420 g trisodium citrate;
the gelling agent, wherein the gelling agent is selected from the group consisting of hydroxyethyl cellulose, oligomers of cellulose, pectin, carboxymethyl cellulose, guar gum, gum arabic, and mixtures thereof; and
balance water from the sterilized and deionized source.

9. A method for mitigating a burn injury to a burn victim using a gel formulation according to claim 1, the method comprising:
applying the gel formulation within 10 minutes of a burn injury on injured skin of the burn victim;
spreading the applied gel formulation on the injured skin to prevent loss of vascular fluid into extracellular regions, to expedite in situ sodium-ion transfer across transdermal membranes in vivo, to in situ expel potassium ions across the transdermal membranes in vitro, and to prevent blister formation or proliferation; and
reapplying fresh gel formulation on the injured skin to maintain high sodium ion concentration gradient across transdermal membranes in vitro to in vivo and high potassium ion concentration gradient across the transdermal membranes in vivo to in vitro.

10. The method of claim 9, wherein the gel formulation has a pH from 7.01 to 10.00, a yield point of greater than or equal to 1000 poise, and an apparent viscosity from greater than 100 centipoise to 150,000 centipoise, the gel formulation comprising, per liter of the gel formulation at 25° C.:
from 80 g to 340 g sodium chloride;
from 6 × 10⁻⁵ g to 42 g sodium bicarbonate;
from 1.0 × 10⁻⁶ g to 1.3 g sodium carbonate;
from 1.6 × 10⁻² g to 156 g sodium lactate;
from 1.53 × 10⁻³ g to 82 g sodium acetate;
from 0.198 g to 420 g trisodium citrate; and
the gelling agent, wherein the gelling agent is selected from the group consisting of hydroxyethyl cellulose, oligomers of cellulose, pectin, carboxymethyl cellulose, guar gum, gum Arabic, and mixtures thereof.

11. The method of claim 9, wherein all salts present in the gel formulation are sodium salts and the gel formulation does not contain any potassium salts or potassium ions.

12. The method of claim 9, wherein the gel formulation has a pH from 7.45 to 10.00, a yield point of greater than or equal to 1000 poise, and an apparent viscosity from greater than 100 centipoise to 150,000 centipoise, the gel formulation consisting essentially of, per liter of the gel formulation at 25° C.:
from 300 g to 340 g sodium chloride;
from 3.5 × 10⁻⁴ g to 42 g sodium bicarbonate;
from 1 × 10⁻⁶ g to 1.3 g sodium carbonate;
from 1.2 × 10⁻¹ g to 156 g sodium lactate;
from 1.15 × 10⁻² g to 82 g sodium acetate;
from 1.471 g to 420 g trisodium citrate;
the gelling agent, wherein the gelling agent is selected from the group consisting of hydroxyethyl cellulose, oligomers of cellulose, pectin, carboxymethyl cellulose, guar gum, and gum arabic; and
balance water from a sterilized and deionized source.

13. The method of claim 9, wherein the gel formulation comprises:
sodium chloride in an amount sufficient to mitigate hyponatremia in blister fluids, extracellular fluids, and blood plasma with simultaneous pain management while restoring sodium/potassium ion imbalances from the burn injury;
sodium bicarbonate in an amount sufficient to result in mitigating respiratory and metabolic acidosis (in situ) and SID in blister fluids (in situ) when pH drops below 7.35 in extracellular fluid and blood plasma with simultaneous pain management while restoring sodium/potassium ion imbalances from the burn injury;
sodium lactate in an amount sufficient to result in mitigating metabolic acidosis and SID management in blister fluid (in situ), extracellular fluid, and blood plasma; and
gelling agent in an amount sufficient to prevent hyperkalemia and acidosis in blister fluids, extracellular fluids, and blood plasma by receiving in vitro excess K⁺ and H⁺ ions from blister fluids, blood plasma, extracellular fluid (in situ), while (in situ) delivering hydroxyl (OH⁻) ions from the gel formulation in vivo into the blister fluid, extracellular fluid, and blood plasma to prevent acidosis and sodium ions (Na⁺) from the gel formulation in vivo into the blister fluid, extracellular fluid, and blood plasma to prevent hyponatremia,
whereby:
the combination of water, sodium chloride, sodium bicarbonate, sodium lactate, and gelling agent in the gel formulation simultaneously rectifies pH imbalances due to respiratory and metabolic acidosis, in situ expels excess K⁺ ions in vitro, in situ repletes Na⁺ ion deficiency in vivo, in situ restores dynamic physiological Na⁺/K⁺ ion imbalances, and mitigates SID imbalances within blister fluid, extracellular fluid and blood plasma/serum with simultaneous pain management while restoring sodium/potassium ion imbalances from the burn injury.

14. A burn treatment kit comprising:
a first gel formulation that mitigates acidosis, hyponatremia, hyperkalemia when applied following a burn injury; and
a second gel formulation that mitigates alkalosis, hyponatremia, hyperkalemia after blister formation is apparent after the burn injury.

15. The burn treatment kit of claim 14, wherein:
the first gel formulation comprises, per liter of the first gel formulation at 25° C.:
from 300 g to 340 g sodium chloride;
from 3.5 × 10⁻⁴ g to 42 g sodium bicarbonate;
from 1 × 10⁻⁶ g to 1.3 g sodium carbonate;
from 1.2 × 10⁻¹ g to 156 g sodium lactate;
from 1.15 × 10⁻² g to 82 g sodium acetate; and
from 1.471 g to 420 g trisodium citrate;
a gelling agent selected from the group consisting of hydroxy ethyl cellulose, oligomers of cellulose, pectin, carboxy-methyl cellulose, guar gum, and gum arabic, and combinations thereof; and
water from a sterilized and deionized source;
the first gel formulation has:
a pH from 7.45 to 10;
a total sodium-ion concentration greater than or equal to 154 g/L;
a total bicarbonate-ion concentration from 0.01 g/L to 17.70 g/L;
a yield point of greater than or equal to 1000 poise; and
an apparent viscosity from greater than 100 centipoise to 150,000 centipoise;
the second gel formulation comprises, per liter of the second gel formulation at 25° C.:
from 80 g to 300 g sodium chloride;
from 6.0 × 10⁻⁵ g to 17.7 g sodium bicarbonate;
from 1.0 × 10⁻⁶ g to 2.3 × 10⁻⁴ g sodium carbonate;
from 1.6 × 10⁻² g to 7.67 × 10⁻² g sodium lactate;
from 1.53 × 10⁻³ g to 7.2 × 10⁻² g sodium acetate;
from 0.198 g to 0.954 g trisodium citrate;
a gelling agent selected from the group consisting of hydroxy ethyl cellulose, oligomers of cellulose, pectin, carboxy-methyl cellulose, guar gum, gum arabic, and combinations thereof; and
water from a sterilized and deionized source; and
the second gel formulation has:
a pH from 7.01 to 7.35;
a total sodium-ion concentration greater than or equal to 120 g/L;
a total lactate-ion concentration from 0.01 g/L to 0.08 g/L;
a yield point of greater than or equal to 1000 poise; and
an apparent viscosity of less than or equal to 150,000 centipoise.

16. The burn treatment kit of claim 15, wherein all salts present in the first gel formulation and all salts present in the second gel formulation are sodium salts and the gel formulations do not contain any potassium salts or potassium ions.

17. A method for mitigating burn injuries to a human using the burn treatment kit according to any of claim 14, the method comprising:
applying the first gel formulation to a human having acidosis, hyponatremia, hyperkalemia in extracellular blister fluid within 10 minutes after a burn injury occurs;
applying the second gel formulation to the human having alkalosis, hyponatremia, hyperkalemia to a blister that becomes prominent after ten minutes,
wherein:
application of the first gel formulation results in mitigation of acidosis, hyponatremia, hyperkalemia; and
application of the second gel formulation results in mitigation of alkalosis, hyponatremia, hyperkalemia after blister formation is visible.

18. The method of claim 17, wherein:
the first gel formulation comprises, per liter of the first gel formulation at 25° C.:
from 300 g to 340 g sodium chloride;
from 3.5 × 10⁻⁴ g to 42 g sodium bicarbonate;
from 1 × 10⁻⁶ g to 1.3 g sodium carbonate;
from 1.2 × 10⁻¹ g to 156 g sodium lactate;
from 1.15 × 10⁻² g to 82 g sodium acetate; and
from 1.471 g to 420 g trisodium citrate;
a gelling agent selected from the group consisting of hydroxy ethyl cellulose, oligomers of cellulose, pectin, carboxy-methyl cellulose, guar gum, and gum arabic, and combinations thereof; and
water from a sterilized and deionized source;
the first gel formulation has:
a pH from 7.45 to 10;
a total sodium-ion concentration greater than or equal to 154 g/L;
a total bicarbonate-ion concentration from 0.01 g/L to 17.70 g/L;
a yield point of greater than or equal to 1000 poise; and
an apparent viscosity from greater than 100 centipoise to 150,000 centipoise;
the second gel formulation comprises, per liter of the second gel formulation at 25° C.:
from 80 g to 300 g sodium chloride;
from 6.0 × 10⁻⁵ g to 17.7 g sodium bicarbonate;
from 1.0 × 10⁻⁶ g to 2.3 × 10⁻⁴ g sodium carbonate;
from 1.6 × 10⁻² g to 7.67 × 10⁻² g sodium lactate;
from 1.53 × 10⁻³ g to 7.2 × 10⁻² g sodium acetate;
from 0.198 g to 0.954 g trisodium citrate;
a gelling agent selected from the group consisting of hydroxy ethyl cellulose, oligomers of cellulose, pectin, carboxy-methyl cellulose, guar gum, gum arabic, and combinations thereof; and
water from a sterilized and deionized source; and
the second gel formulation has:
a pH from 7.01 to 7.35;
a total sodium-ion concentration greater than or equal to 120 g/L;
a total lactate-ion concentration from 0.01 g/L to 0.08 g/L;
a yield point of greater than or equal to 1000 poise; and
an apparent viscosity of less than or equal to 150,000 centipoise.

19. The method of claim 18, wherein all salts present in the first gel formulation and all salts present in the second gel formulation are sodium salts, and the gel formulations do not contain any potassium salts or potassium ions.

20. The method of claim 18, wherein:
the first gel formulation comprises sodium chloride in an amount sufficient to mitigate hyponatremia in blister fluids, extracellular fluids, and blood plasma with simultaneous pain management while restoring sodium/potassium ion imbalances from the burn injury;
the first gel formulation comprises sodium bicarbonate in an amount sufficient to result in mitigating respiratory and metabolic acidosis and SID in blister fluids when pH drops below 7.35 in extracellular fluid and blood plasma with simultaneous pain management while restoring sodium/potassium ion imbalances from the burn injury;
the first gel formulation comprises sodium lactate in an amount sufficient to result in mitigating metabolic acidosis and SID management in blister fluid, extracellular fluid, and blood plasma;
the first gel formulation comprises gelling agent in an amount sufficient to prevent hyperkalemia and acidosis in blister fluids, extracellular fluids, and blood plasma by in situ receiving in vitro excess K⁺ and H⁺ ions from blister fluids, blood plasma, extracellular fluid, while in situ delivering hydroxyl ions (OH⁻) from the gel formulation in vivo into the blister fluid, extracellular fluid, and blood plasma to prevent acidosis and sodium ions (Na⁺) from the gel formulation in vivo into the blister fluid, extracellular fluid, and blood plasma to prevent hyponatremia;
the second gel formulation comprises sodium chloride in an amount sufficient to result in mitigating hyponatremia in blister fluid, extracellular fluid and blood plasma with simultaneous pain management while restoring sodium/potassium ion imbalances from the burn injury;
the second gel formulation comprises sodium lactate and lactic acid in an amount sufficient to result in mitigating alkalosis when plasma pH increases above 7.45 and SID management in blister fluid, extracellular fluid and blood plasma with simultaneous pain management while restoring sodium/potassium ion imbalances from the burn injury; and
the second gel formulation comprises gelling agent in an amount sufficient to result in preventing hyperkalemia (alkalosis) in blister fluid, extracellular fluid and blood plasma by receiving (in vitro) excess K⁺ ion in situ by in situ delivering the stored sodium (Na⁺) ions in vivo in blister fluid, extracellular fluid and blood plasma,
whereby:
in the first gel formulation, the combination of water, sodium chloride, sodium bicarbonate, sodium lactate, and gelling agent in the gel formulation simultaneously rectifies pH imbalances in situ due to respiratory and metabolic acidosis, expels excess K⁺ ions in vitro, in situ replete Na⁺ ion deficiency in vivo, restores dynamic physiological Na⁺/K⁺ ion imbalances, and mitigates SID imbalances within blister fluid, extracellular fluid and blood plasma/serum with simultaneous pain management while restoring sodium/potassium ion imbalances from the burn injury; and
in the second gel formulation, the combination of water, sodium chloride, sodium bicarbonate, sodium lactate, and gelling agent simultaneously rectifies pH imbalances due to respiratory and metabolic alkalosis, in situ expels excess K⁺ ions in vitro, in situ repletes Na⁺ ion deficiency in vivo by restoring dynamic physiological Na⁺/K⁺ ion imbalances and SID imbalances within blister fluids, extracellular fluids and blood plasma/serum with simultaneous pain management while restoring sodium/potassium ion imbalances from the burn injury.
